(19) United States
(12) Patent Application Publication
(10) Pub. No.: US 2016/0095943 A1
(43) Pub. Date: Apr. 7, 2016

(54) Visualization of Foreign Bodies in Tissue

(71) Applicant: Ken Vanderbeck, San Diego, CA (US)

(72) Inventor: Ken Vanderbeck, San Diego, CA (US)

(21) Appl. No.: 14/866,543

(22) Filed: Sep. 25, 2015

Related U.S. Application Data
(60) Provisional application No. 62/059,051, filed on Oct. 2, 2014.

(51) Int. Cl. A61K 49/00; A61B 5/00

(52) U.S. Cl. CPC A61K 49/0043; A61B 5/0071

(57) ABSTRACT

Agents and methods for visualization and removal of foreign material from tissue have been described. The techniques include selection of an appropriate visualization agent, treating the wound with the visualization agent and observing the wound, illuminating the wound with lighting specific to the particular visualization agent and material and viewing the illuminated wound through an optical filter specific to the wavelength of light emitted by the visualization agent.

### CROSS-REFERENCE TO RELATED APPLICATIONS

This application claims priority to U.S. Provisional application 62/059051, titled: Visualization of Foreign Bodies in Tissue, filed on Oct. 2, 2014, by the same inventor and currently pending.

### BACKGROUND OF THE INVENTION

1. Technical Field The present invention relates to methods for visualizing foreign bodies in tissue.

2. Related Background Art

Removing foreign bodies from tissue is a common emergency room procedure. Injuries inflicted on both humans and other animals from glass, wood, metal, plastic and other materials are common reasons for a visit to the emergency room, doctor or hospital. Frequently the foreign material or particulate fractions thereof that caused the injury remain in the wound and must be removed. Foreign bodies left in wounds can cause long-term health problems including inflammation and infections. Finding the foreign material in the wound amongst the tissue, blood and body fluid is difficult. Various visualization techniques such as x-ray, magnetic resonance, computed tomography imaging (CT) and sonography have been explored for enhanced visualization of foreign bodies. These techniques have had limited success and suffer from the requirement of additional often expensive instrumentation and preparation of the patient causing lost time in what may be an emergency situation. Frequently the visualization techniques are specific to a particular type of material and the required equipment is not often available in an emergency room and never available where treatment is taking place in the field, that is outside of a controlled medical room treatment room environment. There is a need for a system that allows for visualization of foreign bodies in human and animal tissue that requires a minimum of additional equipment, is non-toxic to the patient and can be used both in a hospital environment and elsewhere.

### DISCLOSURE OF THE INVENTION

The invention addresses the shortcomings of the present methods by providing methods for visualization of foreign material in animal tissue. The invented system has been found to be especially effective in visualize glass particulate embedded in human tissue. The system includes cleaning of the wound as is normally done, treatment of the wound with a visualization agent and observation of the wound after treatment with lighting to enhance the contrast between the foreign material and the tissue. In one embodiment the method includes fluorescence visualization agents that cause the foreign material to fluoresce and human tissue does not fluoresce under the lighting and thereby increase the contrast between the foreign material and the human tissue. In another embodiment the method includes use of fluorescence visualization agents that cause the human tissue to fluoresce while the foreign material does not fluoresce and thereby increasing the contrast between the foreign material and the human tissue. In one embodiment the fluorescence visualization agent is fluorescein and the foreign material is glass and the treatment results in a fluorescent glow from the foreign glass material in a wound where the human tissue treated by the proscribed procedure does not fluoresce. The glass is seen to be easily visualized under UV illumination. Although examples are exclusively human tissue and wounds in human tissue the techniques are equally applicable to any animal tissue.

### BRIEF DESCRIPTION OF THE DRAWINGS

FIG. 1 is an image of a wound before treatment.

FIG. 2 is an image of the wound in FIG. 1 illuminated with a UV light.

FIG. 3 is an image of the wound of FIGS. 1 and 2 after treatment with a fluorescing visualization agent and illuminated with a UV light.

FIG. 4 is a view of the foreign material glass fragment removed from the wound illuminated under ambient light and not fluorescing.

FIG. 5 is a flow chart for the method.

### MODES FOR CARRYING OUT THE INVENTION

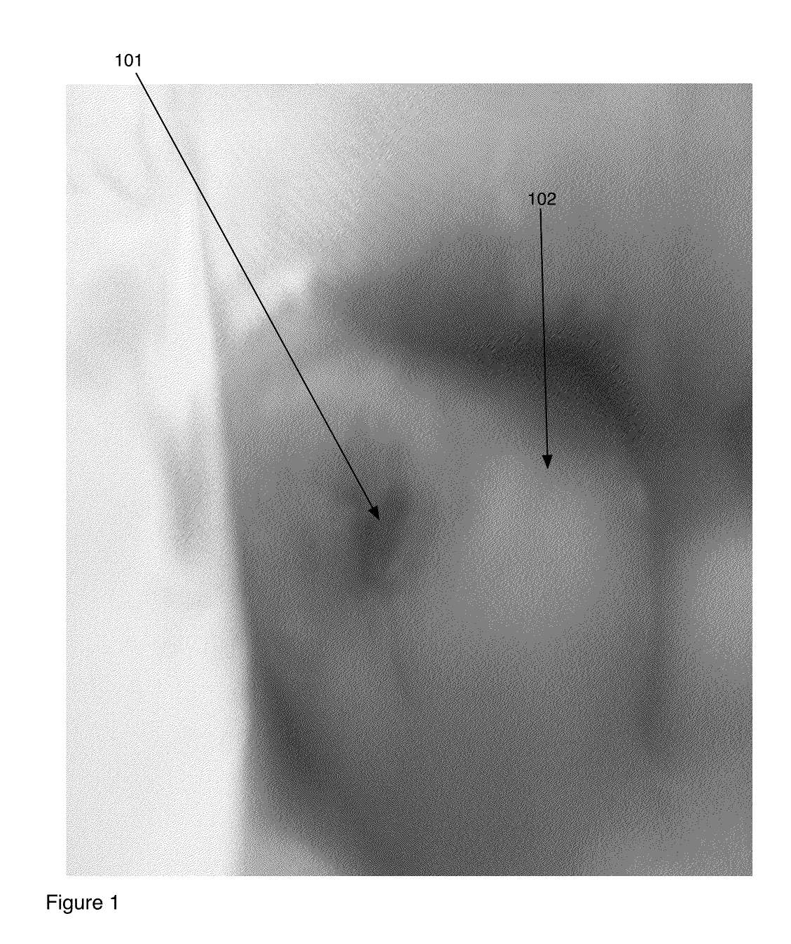

Referring now to FIG. 1, an image of a puncture wound 101 in the toe 102 of a human is shown. Note that although the wound is clearly visible and there is darkening within the wound due to blood. It is not possible to see any foreign material within the wound itself. It is not possible to see the material that may have caused the puncture wound and that is still likely to be embedded in the toe.

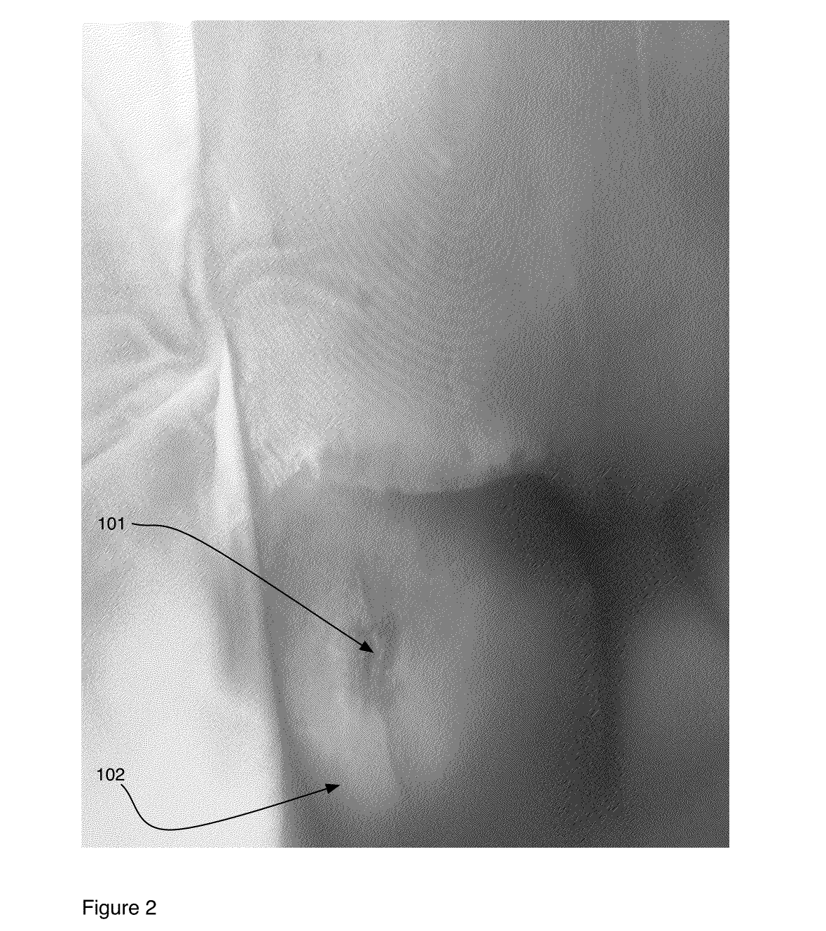

Referring to FIG. 2 the same puncture wound as shown in FIG. 1 is shown, but now illuminated with a UV light in hopes of increasing contrast between the tissue and potential foreign matter embedded in the wound. As before, although the wound is clearly visible and there is darkening within the wound due to blood. It is not possible to see any foreign material within the wound itself. It is not possible to see the material that may have caused the puncture wound and that is still likely to be embedded in the toe.

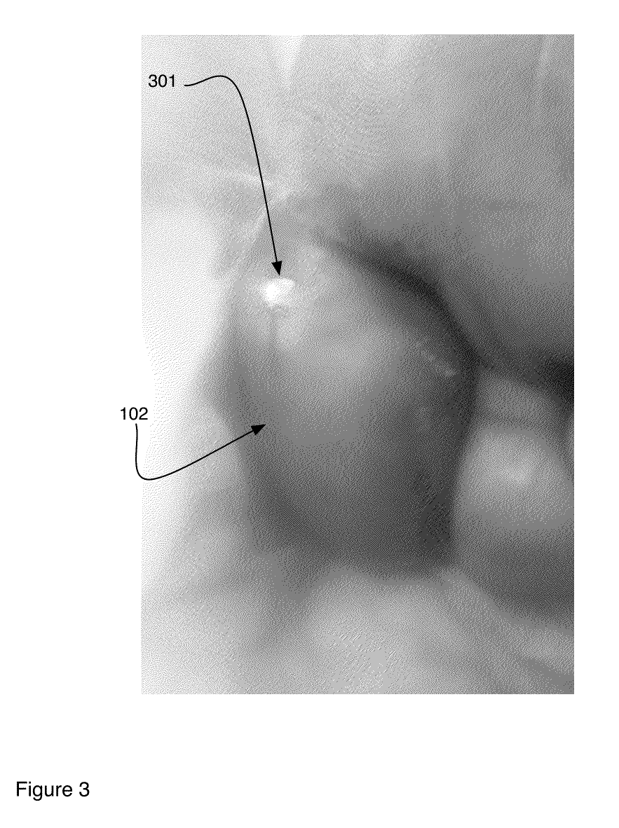

FIG. 3 shows the same puncture wound as in the previous FIGS. 1-2 but now after treatment of the wound with a visualization agent and illumination under UV light. The foreign matter within the wound 301 is seen to glow brightly. It is easily spotted for removal. In the instant case the visualization agent used was fluorescein. The foreign matter is a piece of glass.

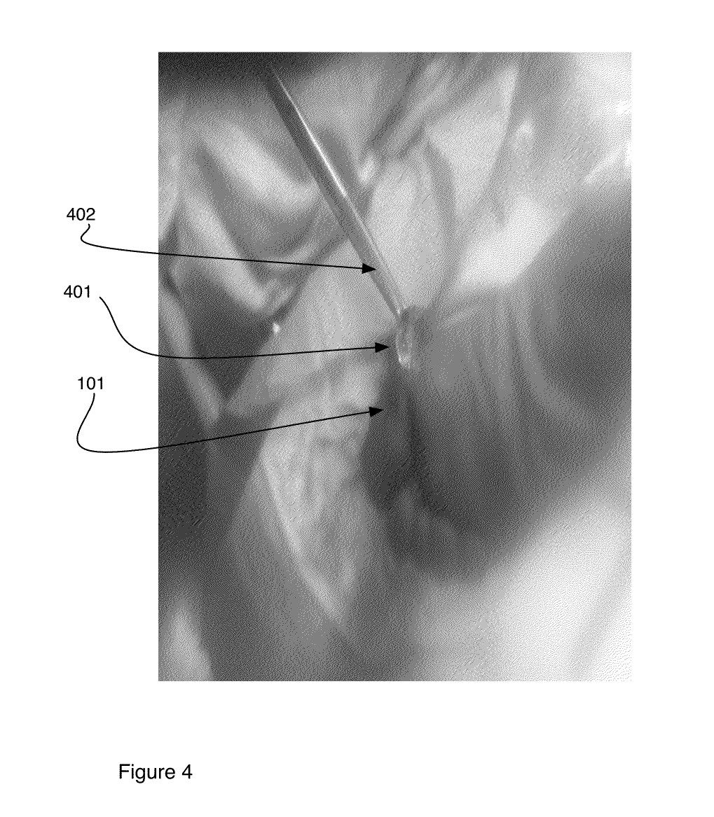

FIG. 4 shows the glass foreign material 401 held in forceps 402 after removal from the puncture wound 101 in the toe 102. In this image the glass and wound are illuminated under normal indoor lighting. Once removed the foreign material is seen to be relatively large. Clearly the bulk of the glass shard was buried in the toe and through the visualization technique the tip of the shard became visible to allow removal.

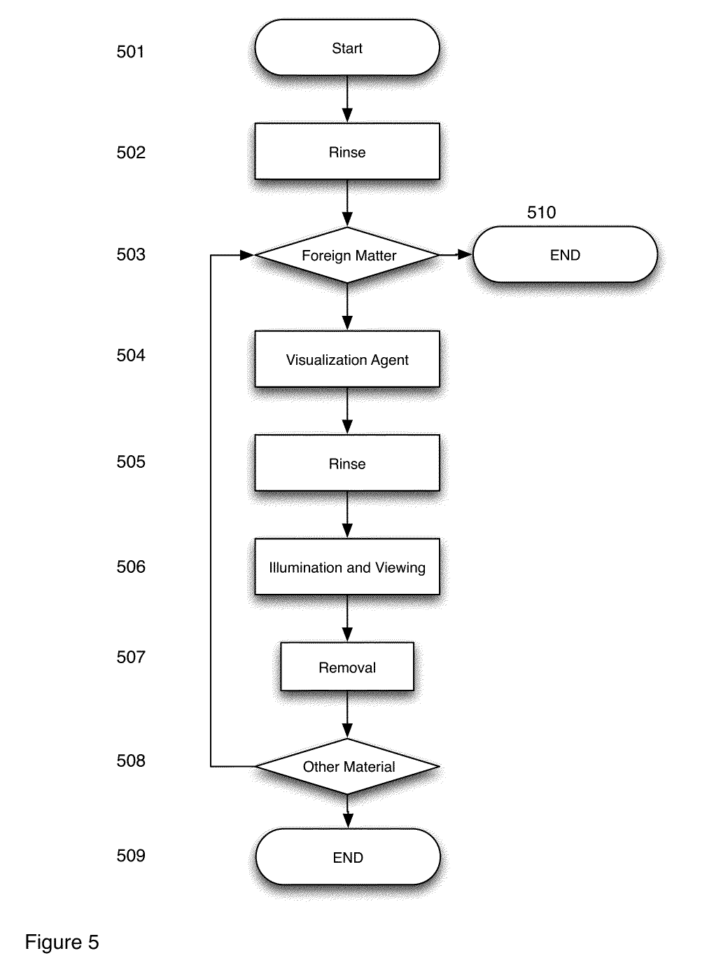

FIG. 5 shows a general procedure for treatment of wounds and the invented techniques for visualization of foreign material. The patient arrives 501 for treatment either at the scene of an injury or the patient showing up at the doctor's office or emergency room. The patient issue is a wound and the wound is first rinsed 502, typically with sterile saline as is known in the art. If either through observation of the wound or other information, such as disclosure from the patient or other persons accompanying the patient, it becomes evidence that there is or could be foreign material in the wound a decision 503 is made as to whether there is likely foreign material and the likely type or types of foreign material. If the conclusion is that there is no foreign material in the wound the procedure with respect to visualization of foreign materials is ended 510. The decision further includes the type of suspected foreign material and from the type of foreign material a particular visualization agent is selected. Table 1 shows the information used for selection of the appropriate visualization agent and lighting. For example, for glass, Fluorescein is the selected visualization agent and the glass can be seen when the wound with the glass is illuminated with UV light as demonstrated in FIGS. 1-4. In another embodiment the tissue is stained rather than the foreign material. In the case of a non-ferromagnetic material where the material is inert to common safe stains, a stain that will stain protein and other body tissue material and not stain the foreign body non-ferromagnetic material is selected. The stain selective adhered to the tissue enhances the contrast between the tissue and the unstained foreign material. Non-limiting examples of stains that are used include Eosin dye, propidium iodide, Trypan Blue, Erythrosine and 7-Aminaoactinomycin D. In the example of propidium iodide the excitation maximum for fluorescence is at 535 nm and the emission maximum is at 617 nm. The illumination and viewing sources in the preferred embodiment include an illumination source with power covering the excitation maximum. Nonlimiting examples for illumination include a xenon arc lamp, a mercury arc lamp and the 4880 line from an argon ion laser. Viewing is made specific by using a filter for the 617 nm emission. Ferromagnetic metal can be “visualized” and removed with the insertion of a small magnet into or near the wound tissue.

In another embodiment to visualize glass the visualization agent is Fluorescein illuminated with UV light with energy available at 494 nm the peak excitation wavelength for fluorescein and viewed with a 521 nm interference filter.

TABLE 1
Visualization Agents

| Type of Foreign | | | |
|---|---|---|---|
| Material | Visualization Agent | Illumination | Viewing |
| Glass | Fluorescein | UV Light | No Filter |
| Glass | Fluorescein | UV Light | 521 nm interference |
| | | | filter |
| Wood | | | |
| Plastic | | | |
| Miscellaneous | Acridine Orange | UV/Blue Light | 525 nm interference |
| | | | filter |
| Metal - non- | Vital Stain (see text) | Specific to the Stain | Specific to the stain |
| ferromagnetic | | | |
| Metal non- | Propidium iodide | Xenon, mercury arc or | 617 nm interference |
| ferromagnetic | | argon ion 4880 line | filter |
| Metal - Ferromagnetic | Magnet | N/A, material adheres | |
| | | to magnet | |

The selected visualization agent is applied 504 and the wound is then rinsed 505 of excess visualization agent and illuminated 506 with an illumination selected for the particular visualization agent. In one embodiment illumination includes both a selection of the wavelength or wavelength range of the source for the lighting and a selection of an optical filter through which the treated tissue is viewed. The use of the visualization agent and the selected illumination allows the doctor or other person provided treatment to see and remove 507 the foreign material. Once removed a second decision 508 is made as to whether there is other types of foreign material potentially in the wound. If so the procedure routes to the step already described 503 as to decision regarding foreign material and the selection of the appropriate visualization agent.

In the specific example of FIGS. 1-4 the procedure of FIG. 5 used in conjunction with Table 1 is more specifically:
The patient arrived at emergency room and indicated they had stepped on glass.
The wound is initially irrigated with 10 cc 0.9% normal saline solution.
Glass is at least a first known potential foreign material and fluorescein is the chosen visualization agent.
Approximately 10 cc of a 10% fluorescein solution is used to irrigate the wound.
The wound is then again irrigated with 10 cc of normal saline.
A small penlight black light (UV Source) is then used to illuminate the wound.
Any fluorescing foreign bodies are then removed using forceps.
No other foreign material is suspected the procedure with respect to visualization of the foreign bodies in tissue is complete.

If other foreign bodies had been suspected the procedure would have looped back to the step of selecting the appropriate visualization agent for the particular foreign body material.

In another embodiment the visualization agent is formulated with sterilization agents or bacteriocides used for cleansing wounds. Nonlimitining examples of bacteriocides include polyaminopropyl biguanide and chemical complexes of polyvinylpyrrolidone and elemental iodine such as sold under the brand betadine ® (Betadine is a registered trademark of Purdue Products L.P. of Delaware US). In one embodiment the visualization agent is an aqueous solution that includes 10% by weight fluorescein and 10% by weight of a polyvinylpyrrolidone iodine complex. The solution is used in the procedure of FIG. 5 and is used to irrigate the wound instead of the 10 cc of 10% fluorescein solution. The solution is used in one embodiment with UV illumination and no filtering for viewing and in another embodiment with UV illumination and viewed through a 521 nm filter for enhanced contrast between the foreign material and the tissue. The solution provides enhanced visualization of foreign bodies in the tissue of the wound.

In another embodiment the visualization agent is an aqueous solution that includes 0.02 mg/ml polyaminopropyl biguanide and 10% fluorescein. The solution is used in the procedure of FIG. 5 and is used to irrigate the wound instead of the 10 cc of 10% fluorescein solution. The solution is used in one embodiment with UV illumination and no filtering for viewing and in another embodiment with UV illumination and viewed through a 521 nm filter for enhanced contrast between the foreign material and the tissue. The solution provides enhanced visualization of foreign bodies in the tissue of the wound.

### SUMMARY

Agents and methods for visualization and removal of foreign material from tissue have been described. The techniques include selection of an appropriate visualization agent, treating the wound with the visualization agent and observing the wound with lighting specific to the particular visualization agent and material.

Those skilled in the art will appreciate that various adaptations and modifications of the preferred embodiments can be configured without departing from the scope and spirit of the invention. Therefore, it is to be understood that the invention may be practiced other than as specifically described herein, within the scope of the appended claims.

What is claimed is:

1. A method for visualization and removal of foreign material from animal tissue said method comprising:
a) rinsing of the animal tissue with sterile saline solution,
b) selecting a fluorescing visualization agent that is specific to the type of foreign material, said visualization agent at least one selected from: fluorescein and propidium iodide,
c) selecting an illumination source that radiates with power over a wavelength that includes the fluorescence excitation maximum for the visualization agent,
d) selecting a viewing light filter that excludes light outside of the fluorescence emission maximum for the visualization agent,
e) treating the animal tissue that includes the foreign material with the visualization agent,
f) viewing the tissue with the foreign material using the illumination source and the viewing light filter thereby seeing the foreign material with an enhanced contrast,
g) removing the foreign material.

2. The method of claim 1 where the visualization agent is one selected from:
a) a 10% by weight aqueous solution of fluorescein,
b) a 10% by weight aqueous solution of propidium iodide,
c) an aqueous solution comprising 10% by weight fluorescein and 10% by weight of a polyvinylpyrrolidone iodine complex,
d) an aqueous solution that includes that includes 0.02 mg/ml polyaminopropyl biguanide and 10% by weight fluorescein.

3. An agent for visualization of foreign material in a wound in animal tissue said agent comprising at least one selected from:
a) a 10% by weight aqueous solution of fluorescein,
b) a 10% by weight aqueous solution of propidium iodide,
c) an aqueous solution comprising 10% by weight fluorescein and 10% by weight of a polyvinylpyrrolidone iodine complex,
d) an aqueous solution that includes that includes 0.02 mg/ml polyaminopropyl biguanide and 10% by weight fluorescein.
